(19) United States
(12) Patent Application Publication
(10) Pub. No.: US 2015/0068317 A1
(43) Pub. Date: Mar. 12, 2015

(54) TEST APPARATUS WITH GIMBALED MOUNT FOR CATHETERS

(71) Applicant: BIOTRONIK SE & Co. KG, Berlin (DE)

(72) Inventors: Joerg Feldmann, Berlin (DE); Heinrich Widdecke, Potsdam (DE); Gernot Kolberg, Berlin (DE)

(21) Appl. No.: 14/477,992

(22) Filed: Sep. 5, 2014

Related U.S. Application Data
(60) Provisional application No. 61/876,771, filed on Sep. 12, 2013.

(51) Int. Cl. G01N 3/08; A61B 19/00

(52) U.S. Cl. CPC G01N 3/08; A61B 19/46; A61B 2019/464
USPC 73/818

(57) ABSTRACT

A test apparatus for performing compression tests on catheters. In order to perform the compression tests in a controlled manner and without the risk of causing damage to the catheter, the test apparatus in accordance with the present invention has a gimbal, to which a mount for the catheter is attached.

### CROSS-REFERENCE TO RELATED APPLICATIONS

This patent application claims the benefit of co-pending U.S. Provisional Patent Application No. 61/876,771, filed on Sep. 12, 2013, which is hereby incorporated by reference in its entirety.

### TECHNICAL FIELD

The present invention relates to a test apparatus for performing compression tests on catheters and, more particularly, to a test apparatus comprising a mount having a receiving opening for an end of the catheter.

### BACKGROUND

When performing compression tests, catheters are inserted via an end and, for example, via the tip of the catheter into the receiving opening and are pressed into this opening for compression. In so doing, the catheters bend away laterally in an undefined manner and are thus arranged in the receiving opening in an undefined manner, whereby the result of the compression test is influenced. Further, the catheter may slip out from the receiving opening, whereby the catheter and, in particular, an electrode tip can be damaged from the screw electrode thereof. If the tip is fixed, for example, by screwing the screw electrode into a mass that can be punctured by the fixing helix, the electrode tip and, in particular, an optionally polished section of the electrode tip, may thus be damaged.

The present invention is directed toward overcoming one or more of the above-identified problems.

### SUMMARY

An object of the present invention is, therefore, to provide a test apparatus with which compression tests can be performed efficiently and expediently on catheters without, on the one hand, damaging the catheter to be tested and, on the other hand, providing the individual curvature characteristics inherent through the catheter with a mount that can tilt freely in all directions.

For the test apparatus mentioned at the outset, at least the object is achieved in that the test apparatus has a gimbal, in which the mount is mounted pivotably about two axes of rotation. Due to the fact that the suspension is mounted pivotably about two axes, it can follow any arbitrary lateral bending of the catheter during the compression test and can securely hold the catheter tip, even with relatively large lateral evasive movements of the catheter. The catheter tip is thus reliably prevented from slipping out from the receiving opening. During the compression test the possible movement of the catheter is thus limited to bends about a predetermined pivot point determined by the axes of rotation. Due to the freely movable receiving opening, the electrode can curve in an unimpeded manner depending on the stability thereof. The actual conditions in the heart are thus reproduced to the greatest extent possible by this suspension.

Usefulness of the present invention lies at least in the following points:
1. There is a mount for the catheter tip, which mount can be tilted in all directions horizontally relative to the vertical direction of insertion of the catheter.
2. Due to this technical prerequisite, the plane of the stop face of the mount adapts 100% to the specific curvature behavior of the catheter during the feed of the catheter, wherein no transverse forces can be produced during the feed by the free movability of the mount.

A solution according to the present invention can be further improved by various embodiments, which are each advantageous per se and can be combined with one another arbitrarily unless specified otherwise. These embodiments and the advantages associated therewith will be discussed hereinafter.

A longitudinal axis of the receiving opening, which can be formed by a bore, may thus run perpendicularly to the axes of rotation. In a predetermined pivot position of the mount, the longitudinal axis thereof may even run perpendicularly to both axes of rotation. The catheter tip can be inserted into the receiving opening along the longitudinal axis, which preferably extends centrally through the receiving opening. Since the longitudinal axis runs perpendicularly to the axes of rotation, the catheter tip pivots about the axes of rotation as the compression test is performed, without a preferred direction.

In order to additionally prevent the mount from rotating in a preferred direction as the compression test is performed, the longitudinal axis may cross the two axes of rotation.

To avoid unwanted forces acting on the mount or the catheter tip, the axes of rotation preferably cross the stop face of the mount.

The receiving opening can be stepped. By way of example, the receiving opening may have a larger diameter transversely to the longitudinal axis thereof in a first region than in a second region adjoining the first region along the longitudinal axis. The stepped receiving opening may thus have a step that can run around the longitudinal axis at least in part or even completely. A face of the step that points against a direction of insertion in which the catheter can be inserted into the receiving opening provides a stop face for the catheter. The catheter can therefore be inserted into the receiving opening to a defined depth. For example, a casing of the catheter may therefore rest against the stop face and is not damaged by the test. The stop face is preferably formed perpendicularly to the longitudinal axis. The second region of the receiving opening, into which the catheter tip may protrude during the compression test, can extend after the stop face in the direction of insertion. Direct contact between the catheter tip and the mount can thus be avoided. As a result, the catheter tip is not damaged during the compression test.

In order to prevent the movement of the mount from having a component in a preferred direction during the compression test, the axes of rotation may cross the step and, particularly, the stop face. Along the longitudinal axis, the catheter thus rests on the step and, in particular, on the stop face provided by the step, wherein the step or the stop face can be arranged in a plane with the axes of rotation. If the step or the stop face were arranged before or after the axes of rotation along the longitudinal axis, a pressure exerted by the catheter onto the step or the stop face would thus cause unwanted rotational movements of the mount.

So that the test apparatus can be used in a versatile manner and with catheters of different dimension, the mount may have an insert element, through which the receiving opening extends at least in part or even completely and which can be repeatedly attached to and removed from a main body of the mount, that is to say can be exchanged. For example, the insert element can be inserted into an opening in the main body and can be removed therefrom. In particular, when the insert element is inserted into the receiving opening, the axes of rotation can run through the plane of the stop face of the mount and/or cross the step or the stop face.

By way of example, the test apparatus may have two or more insert elements with receiving openings of different dimension, wherein the insert elements can be connected to the main body in a manner exchangeable for one another. The receiving openings and, in particular, the first and/or the second region of the receiving openings, preferably have different diameters. Along the longitudinal axis, the step is preferably positioned identically with all insert elements, such that, with use of any one of the insert elements, no unwanted forces or movements with preferred directions are produced which influence the movement of the mount in an undesired and inexpedient manner.

Further features, aspects, objects, advantages, and possible applications of the present invention will become apparent from a study of the exemplary embodiments and examples described below, in combination with the Figure, and the appended claims.

### DESCRIPTION OF THE DRAWINGS

The present invention will be explained hereinafter by way of example on the basis of an embodiment with reference to the drawing. The different features of the embodiments can be combined here independently of one another, as has already been presented in the individual advantageous embodiments. In the drawing:

FIG. 1 shows a schematic illustration of a first exemplary embodiment of the test apparatus according to the present invention.

### DETAILED DESCRIPTION

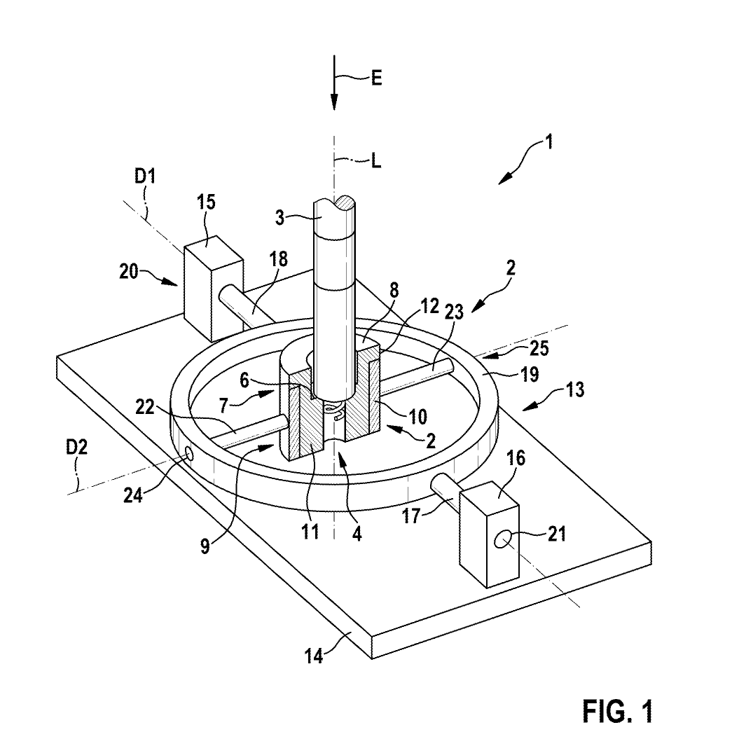

The structure and function of a test apparatus according to the invention will be described hereinafter with reference to the exemplary embodiment of FIG. 1:

FIG. 1 schematically shows a perspective view of the test apparatus 1 for performing compression tests. The test apparatus 1 has a mount 2 for an end of a catheter 3 to be tested. The mount 2 is formed with a receiving opening 4, into which a tip of the catheter 3 can be inserted.

The receiving opening 4 extends along its longitudinal axis L through the mount 2 and is formed, for example, with a circular cross section. Along the longitudinal axis L, an inner diameter of the receiving opening tapers or changes suddenly, such that the receiving opening 4 is formed in a stepped manner. The step 5 formed by the stepped form of the receiving opening 4 provides a stop face 6 for the catheter 3 and, in particular, for a casing of the catheter 3. The stop face 6 is preferably formed horizontally relative to the longitudinal axis L, and the receiving opening 4 runs around the longitudinal axis L.

The catheter 3 can be inserted via the tip thereof into the receiving opening 4 in a direction of insertion arrow E running parallel to the longitudinal axis L. In so doing, the catheter 3 is first inserted into a first region 7 of the receiving opening 4. The first region 7 extends against the direction of insertion arrow E from the step 5 to a longitudinal end 8 of the mount 2. The step 5 and/or the first region 7 is adjoined in the direction of insertion arrow E by a second region 9 of the receiving opening 4, wherein an inner diameter of the second region 9 is smaller than an inner diameter of the first region 7.

The mount 2 may optionally be formed in a number of parts and, particularly, in two parts. By way of example, the mount 2 may have a main body 10, which, for example, can be formed as a sleeve extending along the longitudinal axis L. The mount 2 may also have an insert element 11, which is fastened or can be fastened to the main body 10. If the main body 10 is formed as a sleeve, the insert element 11 can be inserted into the main body 10.

The insert element 11 may comprise the receiving opening 4 and, optionally, also the step 5 or the stop face 6 formed by the step 5. If the insert element 11 is attached to the main body 10 or inserted thereinto, the insert element 11 preferably forms the longitudinal end 8 of the mount 2. Here, the insert element 11 may have a protrusion 12 running transversely relative to the longitudinal axis L and away from the longitudinal axis L, the protrusion 12 preventing the insert element 11 from being inserted too deep into the main body 10. The protrusion 12 may extend completely around the longitudinal axis L.

In the exemplary embodiment of FIG. 1, the receiving opening 4 is formed as a continuous opening. Alternatively, however, the receiving opening 4 and, particularly, the second region 9 of the receiving opening, 4 can also be closed in the direction of insertion E. However, the receiving opening 4 is preferably a continuous opening in order to prevent the catheter tip from colliding with a base of the receiving opening or with contaminations collecting at the base of the receiving opening 4.

In order to prevent the catheter tip from moving in an unwanted manner relative to the mount 2 during the test, the test apparatus 1 has a gimbal 13, in which the mount 2 is suspended rotatably about two axes of rotation D1, D2. The catheter tip of the catheter 3 can thus pivot together with the mount 2 about the axes of rotation D1, D2, such that movements of the catheter 3 occurring during the compression test cannot lead to relative movements between the catheter tip and the mount 2, and also cannot cause the catheter tip to slip out from the mount 2.

The gimbal 13, for example, has two bearing arrangements 15, 16 arranged on a base plate 14 of the test apparatus 1. The bearing arrangements 15, 16 are preferably non-displaceably attached to the base plate 14. For example, rods or bars 17, 18 are attached to the bearing arrangements 15, 16 and each connect one of the bearing arrangements 15, 16 to a bearing ring 19 of the gimbal 13. The axis of rotation D1 extends through the rods 17, 18 and, therefore, the bearing ring 19 is pivotable about the axis of rotation D1. Here, the bearing ring 19 may have bearing elements, which allow a rotation of the bearing ring 19 relative to the rods 17, 18. Alternatively, and as illustrated in the exemplary embodiment of FIG. 1, the bearing arrangements 15, 16 each have a bearing element 20, 21, at which the rods 17, 18 are rotatably mounted about the axis of rotation D1. By way of example, the bearing elements 20, 21 are ball bearings that enable a rotation of the rods 17, 18 about the axis of rotation D1 with little friction. The rods 17, 18 can be fastened immovably to the bearing ring 19.

The mount 2 is preferably rotatably connected via two rods 22, 23 to the bearing ring 19. Here, the rods 22, 23 preferably extend along the axis of rotation D2, such that the mount 2 is rotatable relative to the bearing ring 19 about the axis of rotation D2. The mount 2 may have bearing elements which enable a rotation of the mount 2 about the axis of rotation D2 and relative to the rods 22, 23. As illustrated in the exemplary embodiment of FIG. 1, however, the bearing elements 24, 25 are preferably provided on the bearing ring 19, such that the rods 22, 23 are rotatable relative to the bearing ring 19 about the axis of rotation D2. The rods 22, 23 can be immovably fastened to the mount 2 and in particular to the main body 10 thereof.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. A test apparatus for performing compression tests on catheters, comprising:
a mount having a receiving opening for an end of the catheter; and
a gimbal, in which the mount is mounted pivotably about two axes of rotation.

2. The test apparatus as claimed in claim 1, wherein a longitudinal axis of the receiving opening runs perpendicularly to one of the axes of rotation.

3. The test apparatus as claimed in claim 2, wherein the longitudinal axis crosses the two axes of rotation.

4. The test apparatus as claimed in claim 1, wherein the two axes of rotation cross the stop face of the mount.

5. The test apparatus 1 as claimed in claim 1, wherein the receiving opening is stepped.

6. The test apparatus as claimed in claim 5, wherein the two axes of rotation cross a step of the stepped receiving opening.

7. The test apparatus as claimed in claim 1, wherein the mount has an insert element, through which the receiving opening extends at least in part and which can be repeatedly attached to and removed from a main body of the mount.

8. The test apparatus as claimed in claim 7, wherein the test apparatus has two insert elements with receiving openings of different dimension, wherein the insert elements can be connected to the main body in a manner exchangeable for one another.
